(19) United States
(12) Patent Application Publication
(10) Pub. No.: US 2013/0136785 A1
(43) Pub. Date: May 30, 2013

(54) ACTIVE SUBSTANCE-RELEASING WOUND DRESSING

(71) Applicants: Jan Schönberger, Haan (DE); Michael Mager, Leverkusen (DE); Heike Heckroth, Odenthal (DE); Doris Klee, Aachen (DE); Mona Wambach, Hamburg (DE); Christoph Suschek, Langenfeld (DE)

(72) Inventors: Jan Schönberger, Haan (DE); Michael Mager, Leverkusen (DE); Heike Heckroth, Odenthal (DE); Doris Klee, Aachen (DE); Mona Wambach, Hamburg (DE); Christoph Suschek, Langenfeld (DE)

(73) Assignee: Bayer Intellectual Property GmbH, Monheim (DE)

(21) Appl. No.: 13/578,529

(22) PCT Filed: Feb. 8, 2011
(86) PCT No.: PCT/EP11/51804

§ 371 Date: Jan. 15, 2013

(30) Foreign Application Priority Data
Feb. 11, 2010 (EP) 10001400.0

(51) Int. Cl. A61L 15/26

(52) U.S. Cl. 424/445; 514/565

(57) ABSTRACT

The invention relates to a wound dressing, comprising a liquid-absorbing substrate having active substance depots contained therein, wherein the active substance depots comprise particles of at least one active substance which are encapsulated in a silicone shell. The invention further relates to a wound dressing according to the invention for use as a means for treating wounds and to the use of a wound dressing according to the invention for producing a means for treating wounds.

The invention relates to an active substance-releasing wound dressing.

Use of wound dressings made of foams for treating weeping wounds is prior art. Owing to their high absorbency and their good mechanical properties, polyurethane foams obtainable by reacting mixtures of diisocyanates and polyols or NCO-functional polyurethane prepolymers with water in the presence of certain catalysts and also (foam) additives are used for this in particular. Examples of such wound dressings are described for instance in U.S. Pat. No. 3,978,266, U.S. Pat. No. 3,975,567 and EP-A 0 059 048.

The prior art further includes wound dressings that contain an active substance. These active substances are for example compounds that augment wound healing. The use of active antibacterial or pain-relieving substances is also known. An active substance-containing wound dressing is described for example in WO 2007 068492 A1.

The known wound dressings are mostly saturated/impregnated with the respective active substances. As a consequence, when these wound dressings come to be used, they initially release a large amount of the active substance, but the rate of release then drops off quickly. Yet it would be desirable for the wound dressing to release its active-substance ingredient continuously at a virtually constant rate throughout the entire period of use, since this would optimally augment the healing process. However, this has hitherto not been possible with the known wound dressings especially with active substances that are readily water-soluble.

EP 0 436 729 A1 discloses a bandage that contains active substance-containing microcapsules. The microcapsules in this instance consist of water-resistant thermoset resins. Release of the active substance is accordingly only possible once the microcapsules are destroyed mechanically, for instance by rubbing. At that point, however, the release would be abrupt. There is accordingly no continuous release of the active substance.

The problem addressed by the invention was that of providing a wound dressing capable of ensuring that an active substance contained therein, which may be water-soluble in particular, is released in a continuous manner throughout the entire intended period of use at a substantially consistent rate.

This problem is solved by a wound dressing comprising a liquid-absorbing substrate having active substance depots contained therein, wherein the active substance depots comprise particles of at least one active substance which are encapsulated in a silicone envelope.

The wound dressings of the present invention continuously release in use a substantially constant amount of the active substance from the active substance depots, thereby ensuring that optimal supply of the active substance to the wound is being managed.

In a first preferred embodiment of the invention, the substrate is a foam. It is advantageous here for the substrate to have high absorbency in respect of wound exudates and to be capable of conforming to the wound in a particularly elastic manner.

The foam may more particularly have a density of ≦0.5, preferably of ≦0.4, more preferably of ≦0.01 to ≦0.3 and even more preferably of ≦0.05 to ≦0.3 g/cm³. Wound dressings that include such a foam as substrate are notable for particularly high absorbency and particularly advantageous mechanical properties.

The foam may be a polymer-based foam, preferably a reactive foam or a mechanically blown foam.

In a further preferred embodiment of the invention, the foam is based on polyurethanes.

Suitable polyurethanes are obtainable by reaction of
A) isocyanate-functional prepolymers having a weight fraction of low molecular weight aliphatic diisocyanates having a molar mass of 140 to 278 g/mol of below 1.0 wt %, based on the prepolymer, obtainable by reaction of
   A1) low molecular weight aliphatic diisocyanates having a molar mass of 140 to 278 g/mol with
   A2) di- to hexafunctional polyalkylene oxides having an OH number of 22.5 to 112 mg KOH/g and an ethylene oxide content of 50 to 100 mol %, based on the total amount of oxyalkylene groups present,
B) optionally heterocyclic 4-ring or 6-ring oligomers of low molecular weight aliphatic diisocyanates having a molar mass of 140 to 278 g/mol,
C) water,
D) optionally catalysts,
E) C₈-C₂₂ monocarboxylic acids or their ammonium or alkali metal salts or C₁₂-C₄₄ dicarboxylic acids or their ammonium or alkali metal salts,
F) optionally surfactants,
G) optionally mono- or polyhydric alcohols, and
H) optionally hydrophilic polyisocyanates obtainable by reaction of
   H1) low molecular weight aliphatic diisocyanates having a molar mass of 140 to 278 g/mol and/or polyisocyanates obtainable therefrom with an isocyanate functionality of 2 to 6, with
   H2) monofunctional polyalkylene oxides having an OH number of 10 to 250 and an ethylene oxide content of 50 to 100 mol %, based on the total amount of oxyalkylene groups present.

These polyurethanes make it possible in particular to produce reactive foams which have high absorbency in respect of wound exudates and so are particularly useful as substrate in the wound dressing of the present invention.

The residual monomer content of the prepolymers used in A) is preferably below 0.5 wt %, based on the prepolymer. This level can be achieved via appropriately chosen use levels of A1) and A2). However, it is preferable to use the isocyanate A1) in excess and subsequently remove, preferably by distillation, unconverted monomers.

The isocyanate-functional prepolymers of component A) are typically prepared by reacting one equivalent of polyol component A2), based on the hydroxyl function, with 1 to 20 mol, preferably 1 to 10 mol and more preferably 5 to 10 mol of the low molecular weight aliphatic diisocyanate A1).

The reaction can take place in the presence of urethanization catalysts such as tin compounds, zinc compounds, amines, guanidines or amidines, or in the presence of allophanatization catalysts such as zinc compounds.

The reaction temperatures are typically in the range from 25 to 140° C. and preferably in the range from 60 to 100° C.

When excess isocyanate was used, the excess of low molecular weight aliphatic diisocyanate is thereafter preferably removed by thin film distillation.

Before, during and after the reaction or distillative removal of the excess diisocyanate, acidic or alkylating stabilizers such as benzoyl chloride, isophthaloyl chloride, methyl tosylate, chloropropionic acid, HCl or antioxidants, such as di-tert-butylcresol or tocopherol can be added.

The NCO content of the isocyanate-functional prepolymers A) is preferably in the range from 1.5 to 4.5 wt %, more preferably in the range from 1.5 to 3.5 wt % and most preferably in the range from 1.5 to 3.0 wt %.

Examples of low molecular weight aliphatic diisocyanates of component A1) are hexamethylene diisocyanate (HDI), isophorone diisocyanate (IPDI), butylene diisocyanate (BDI), bisisocyanatocyclohexylmethane (HMDI), 2,2,4-trimethylhexamethylene diisocyanate, bisisocyanatomethylcyclohexane, bisisocyanatomethyltricyclodecane, xylene diisocyanate, tetramethylxylylene diisocyanate, norbornane diisocyanate, cyclohexane diisocyanate or diisocyanatododecane, of which hexa-methylene diisocyanate (HDI), isophorone diisocyanate (IPDI), butylene diisocyanate (BDI) and bis(isocyanatocyclohexyl)methane (HMDI) are preferred. Hexamethylene diisocyanate, isophorone diisocyanate and butylene diisocyanate are particularly preferred and hexamethylene diisocyanate and isophorone diisocyanate are very particularly preferred.

Polyalkylene oxides of component A2) are preferably copolymers of ethylene oxide and propylene oxide having an ethylene oxide content, based on the total amount of oxyalkylene groups present, of 50 to 100 mol %, preferably 60 to 85 mol %, and started on polyols or amines. Suitable starters of this kind are glycerol, trimethylolpropane (TMP), sorbitol, pentaerythritol, triethanolamine, ammonia or ethylenediamine.

The number average molecular weights of the polyalkylene oxides of component A2) are typically in the range from 1000 to 15 000 g/mol and preferably in the range from 3000 to 8500 g/mol.

The polyalkylene oxides of component A2) further have OH functionalities of 2 to 6, preferably of 3 to 6 and more preferably of 3 to 4.

Optional compounds of component B) are heterocyclic 4-ring or 6-ring oligomers of low molecular weight aliphatic diisocyanates having a molar mass of 140 to 278 g/mol such as isocyanurates, iminooxadiazinediones or uretdiones of the aforementioned low molecular weight aliphatic diisocyanates. Heterocyclic 4-ring oligomers such as uretdiones are preferred.

The increased isocyanate group content due to the use of component B) provides better foaming due to more CO₂ formed in the isocyanate-water reaction.

The water used as component C) can be used as such, as water of crystallization of a salt, as solution in a dipolar aprotic solvent or else as an emulsion. Preferably, the water is used as such or in a dipolar aprotic solvent. It is very particularly preferred to use water as such.

To speed urethane formation, component D) may utilize catalysts. The catalysts in question are typically compounds with which a person skilled in the art is familiar from polyurethane technology. Preference here is given to compounds from the group consisting of catalytically active metal salts, amines, amidines and guanidines. Specific examples are dibutyltin dilaurate (DBTL), tin octanoate (SO), tin acetate, zinc octanoate (ZO), 1,8-diazabicyclo[5.4.0]undecene-7 (DBU), 1,5-diazabicyclo[4.3.0]nonene-5 (DBN), 1,4-diazabicyclo[3.3.0]octene-4 (DBO), N-ethylmorpholine (NEM), triethylenediamine (DABCO), pentamethylguanidine (PMG), tetramethylguanidine (TMG), cyclotetramethylguanidine (TMGC), n-decyltetramethylguanidine (TMGD), n-dodecyltetramethylguanidine (TMGDO), dimethylaminoethyltetramethylguanidine (TMGN), 1,1,4,4,5,5-hexamethylisobiguanidine (HMIB), phenyltetramethylguanidine (TMGP) and hexamethyleneoctamethylbiguanidine (HOBG).

Particular preference is given to the use of amines, amidines, guanidines or mixtures thereof as catalysts of component D). Very particular preference is given to using 1,8-diaza-bicyclo[5.4.0]undecene-7 (DBU).

Component E) utilizes ammonium and alkali metal salts of C₈-C₂₂ monocarboxylates or their free carboxylic acids or C₁₂-C₄₄ dicarboxylates or their free dicarboxylic acids, preferably potassium or sodium salts of C₈-C₂₂ monocarboxylates or C₁₂-C₄₄ dicarboxylates and more preferably sodium salts of C₈-C₂₂ monocarboxylates.

Examples of compounds useful as component E) are the ammonium, sodium, lithium or potassium salts of ethylhexanoic acid, octanoic acid, decanoic acid, dodecanoic acid, palmitic acid, stearic acid, the octadecenoic acids, the octadecadienoic acids, the octadecatrienoic acids, isostearic acid, erucic acid, abietic acid and hydrogenation products thereof. Examples of C₁₂-C₄₄ dicarboxylic acids and the ammonium and alkali metal salts derived therefrom are dodecanedioic acid, dodecenylsuccinic acid, tetradecenylsuccinic acid, hexadecenylsuccinic acid, octadecenylsuccinic acid, C₃₆ and C₄₄ dimer fatty acids and hydrogenation products thereof and also the corresponding ammonium, sodium, lithium or potassium salts of these dicarboxylic acids.

Compounds of component F) can be used to improve foam formation, foam stability or the properties of the resulting polyurethane foam, in which case such additives can in principle be any known anionic, cationic, amphoteric and nonionic surfactants and also mixtures thereof. Preference is given to using alkylpolyglycosides, EO-PO block copolymers, alkyl or aryl alkoxylates, siloxane alkoxylates, esters of sulphosuccinic acid and/or alkali or alkaline earth metal alkanoates. Particular preference is given to using EO-PO block copolymers. Preferably, the EO-PO block copolymers are solely used as component F).

In addition, compounds of component G) can be used to improve the foam properties of the resulting polyurethane foam. These compounds comprise in principle any mono- and polyhydric alcohols known per se to a person skilled in the art, and also mixtures thereof. These are mono- or polyhydric alcohols or polyols, such as ethanol, propanol, butanol, decanol, tridecanol, hexadecanol, ethylene glycol, neopentyl glycol, butanediol, hexanediol, decanediol, trimethylolpropane, glycerol, pentaerythritol, monofunctional polyether alcohols and polyester alcohols, polyether diols and polyester diols.

The hydrophilic polyisocyanates H) are typically prepared by adjusting the ratio of monofunctional polyalkylene oxides H2) to low molecular weight aliphatic diisocyanates H1) such that for every 1 mol of OH groups of the monofunctional polyalkylene oxides there are from 1.25 to 15 mol, preferably from 2 to 10 mol and more preferably from 2 to 6 mol of NCO groups of low molecular weight aliphatic diisocyanate H1). This is followed by the allophanatization/biuretization and/or isocyanurate formation/uretdione formation. When the polyalkylene oxides H2) become bonded to the aliphatic diisocyanates H1) via urethane groups, it is preferably an allophanatization which takes place subsequently. It is further preferable for isocyanurate structural units to be formed.

An alternative way to prepare the hydrophilic polyisocyanates H) typically involves reacting 1 mol of OH groups of the monofunctional polyalkylene oxide component H2) with 1.25 to 15 mol, preferably with 2 to 10 mol and more preferably 2 to 6 mol of NCO groups of a polyisocyanate H1) having an isocyanate functionality of 2 to 6, based on aliphatic diisocyanates. Exemplary of such polyisocyanates H1) are biuret structures, isocyanurates/uretdiones based on aliphatic diisocyanates. The polyisocyanates H1) and the polyalkylene oxides H2) are preferably linked together via a urethane group or a urea group, although particularly the linking via urethane groups is preferable.

The reaction can be carried out in the presence of urethanization catalysts such as tin compounds, zinc compounds, amines, guanidines or amidines, or in the presence of allophanatization catalysts such as zinc compounds.

The reaction temperature is typically in the range from 25 to 140° C. and preferably in the range from 60 to 100° C.

When excess low molecular weight diisocyanate was used, excess low molecular weight aliphatic diisocyanate is subsequently removed, preferably by thin film distillation.

Before, during and after the reaction or distillative removal of excess diisocyanate, acidic or alkylating stabilizers, such as benzoyl chloride, isophthaloyl chloride, methyl tosylate, chloropropionic acid, HCl or antioxidants, such as di-tert-butylcresol or tocopherol can be added.

The NCO content (determined to DIN-EN ISO 11909) of the hydrophilic poly-isocyanates H) is preferably in the range from 0.3 to 20 wt %, more preferably in the range from 2 to 10 wt % and most preferably in the range from 3 to 6 wt %.

Examples of low molecular weight aliphatic diisocyanates of component H1) are hexamethylene diisocyanate (HDI), isophorone diisocyanate (IPDI), butylene diisocyanate (BDI), bisisocyanatocyclohexylmethane (HMDI), 2,2,4-trimethylhexamethylene diisocyanate, bisisocyanatomethylcyclohexane, bisisocyanatomethyltricyclodecane, xylene diisocyanate, tetramethylxylylene diisocyanate, norbornane diisocyanate, cyclohexane diisocyanate or diisocyanatododecane, of which hexamethylene diisocyanate (HDI), isophorone diisocyanate (IPDI), butylene diisocyanate (BDI) and bis(isocyanatocyclohexyl)methane (HMDI) are preferable. Hexamethylene diisocyanate (HDI), isophorone diisocyanate (IPDI) and butylene diisocyanate (BDI) are more preferable and hexamethylene diisocyanate (HDI) and isophorone diisocyanate (IPDI) are most preferable.

Examples of comparatively high molecular weight polyisocyanates H1) are polyisocyanates having an isocyanate functionality of 2 to 6 with isocyanurate, urethane, allophanate, biuret, iminooxadiazinetrione, oxadiazinetrione and/or uretdione groups based on the aliphatic and/or cycloaliphatic diisocyanates mentioned in the preceding section.

Preference for use as component H1) is given to comparatively high molecular weight compounds with biuret, iminooxadiazinedione, isocyanurate and/or uretdione groups based on hexamethylene diisocyanate, isophorone diisocyanate and/or 4,4′-diisocyanatodicyclohexylmethane. Isocyanurates are more preferable. Structures based on hexamethylene diisocyanate are most preferable.

Preparing polyalkylene oxides H2) by alkoxylating suitable starter molecules is literature known (e.g., Ullmanns Encyclopädie der technischen Chemie, 4th edition, volume 19, Verlag Chemie, Weinheim pp. 31-38). Suitable starter molecules are especially saturated monoalcohols such as methanol, ethanol, n-propanol, isopropanol, n-butanol, isobutanol, sec-butanol, diethylene glycol monobutyl ether and also aromatic alcohols such as phenol or monoamines such as diethylamine. Preferred starter molecules are saturated monoalcohols of the aforementioned kind. It is particularly preferable to use diethylene glycol monobutyl ether or n-butanol as starter molecules.

Monofunctional polyalkylene oxides for the purposes of the invention are compounds having just one isocyanate-reactive group, i.e., a group capable of reacting with an NCO group.

The monofunctional polyalkylene oxides H2) preferably have an OH group as isocyanate-reactive group.

The monofunctional polyalkylene oxides H2) have an OH number of 15 to 250 and preferably of 28 to 112 and an ethylene oxide content of 50 to 100 mol % and preferably of 60 to 100 mol %, based on the total amount of oxyalkylene groups present.

The number average molecular weights of monofunctional polyalkylene oxides H2) are typically in the range from 220 to 3700 g/mol and preferably in the range from 500 to 2800 g/mol.

Reactive foams are obtainable from the aforementioned polyurethanes by mixing the components A), C) and optionally B), D), E), F), G), H) in any order, foaming the mixture and curing, preferably by chemical crosslinking. The components A), B) and optionally H) are preferably premixed with one another. The components E) and optionally F) can be added to the reaction mixture in the form of their aqueous solutions.

Foaming can in principle be effected by means of the carbon dioxide formed in the course of the reaction of the isocyanate groups with water, but the use of further blowing agents is likewise possible. It is thus also possible in principle to use blowing agents from the class of the hydrocarbons such as C₃-C₆ alkanes, for example butanes, n-pentane, isopentane, cyclopentane, hexanes or the like, or halogenated hydrocarbons such as dichloromethane, dichloromonofluoromethane, chlorodifluoroethanes, 1,1-dichloro-2,2,2-trifluoroethane, 2,2-dichloro-2-fluoro-ethane, particularly chlorine-free hydrofluorocarbons such as difluoromethane, trifluoromethane, difluoroethane, 1,1,1,2-tetrafluoroethane, tetrafluoroethane (R 134 or R134a), 1,1,1,3,3-pentafluoropropane (R 245 fa), 1,1,1,3,3,3-hexafluoropropane (R 256), 1,1,1,3,3-pentafluorobutane (R 365 mfc), heptafluoropropane, or else sulfur hexafluoride. Mixtures of these blowing agents can also be used.

The subsequent curing typically takes place at room temperature.

Alternatively, polyurethanes can be used that are obtainable by
I) isocyanate-functional prepolymers being produced at least from
   I1) organic polyisocyanates,
   I2) polymeric polyols having number average molecular weights of 400 to 8000 g/mol and OH functionalities of 1.5 to 6, and
   I3) optionally hydroxyl-functional compounds having molecular weights of 62 to 399 g/mol, and
   I4) optionally isocyanate-reactive, anionic or potentially anionic and/or optionally nonionic hydrophilicizing agents,
J) their free NCO groups then being wholly or partly reacted
   J1) optionally with amino-functional compounds having molecular weights of 32 to 400 g/mol and/or
   J2) with isocyanate-reactive, preferably amino-functional, anionic or potentially anionic hydrophilicizing agents
by chain extension, and the prepolymers being dispersed in water before, during or after step J), any potentially ionic groups present being converted into the ionic form by partial or complete reaction with a neutralizing agent.

These polyurethanes are especially useful in the form of aqueous dispersions for producing mechanical foams that are likewise notable for high absorbency in respect of wound exudates when used as substrate for the wound dressing of the present invention.

Suitable polyisocyanates of component I1) are the aromatic, araliphatic, aliphatic or cycloaliphatic polyisocyanates of ≧2 NCO functionality that are known per se to a person skilled in the art.

Examples of such suitable polyisocyanates are 1,4-butylene diisocyanate, 1,6-hexamethylene diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or their mixtures of any desired isomer content, 1,4-cyclohexylene diisocyanate, 1,4-phenylene diisocyanate, 2,4- and/or 2,6-tolylene diisocyanate, 1,5-naphthylene diisocyanate, 2,2′- and/or 2,4′- and/or 4,4′-diphenylmethane diisocyanate, 1,3- and/or 1,4-bis(2-isocyanatoprop-2-yl)benzene (TMXDI), 1,3-bis(isocyanatomethyl)benzene (XDI), alkyl 2,6-diisocyanatohexanoate (lysine diisocyanates) with C1-C8 alkyl groups, and also 4-isocyanatomethyl-1,8-octane diisocyanate (nonane triisocyanate) and triphenylmethane 4,4′,4″-triisocyanate.

As well as the aforementioned polyisocyanates, it is also possible to use proportions of modified diisocyanates or triisocyanates of uretdione, isocyanurate, urethane, allophanate, biuret, iminooxadiazinedione and/or oxadiazinetrione structure.

Preferably, the polyisocyanates or polyisocyanate mixtures of the aforementioned kind have exclusively aliphatically and/or cycloaliphatically attached isocyanate groups and an average NCO functionality in the range from 2 to 4, preferably in the range from 2 to 2.6 and more preferably in the range from 2 to 2.4, for the mixture.

It is particularly preferable for I1) to utilize 1,6-hexamethylene diisocyanate, isophorone diisocyanate, the isomeric bis(4,4′-isocyanatocyclohexyl)methanes, and also mixtures thereof.

I2) utilizes polymeric polyols having a number average molecular weight Mn of preferably from 400 to 6000 g/mol and more preferably from 600 to 3000 g/mol.

These preferably have an OH functionality in the range from 1.8 to 3 and more preferably in the range from 1.9 to 2.1.

Such polymeric polyols are the well-known polyurethane coating technology polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols. These can be used in A2) individually or in any desired mixtures with each or one another.

Polyester polyols are for example the well-known polycondensates formed from di- and also optionally tri- and tetraols and di- and also optionally tri- and tetra-carboxylic acids or hydroxycarboxylic acids or lactones. Instead of the free polycarboxylic acids it is also possible to use the corresponding polycarboxylic anhydrides or corresponding polycarboxylic esters of lower alcohols for preparing the polyesters.

Examples of suitable diols are ethylene glycol, butylene glycol, diethylene glycol, triethylene glycol, polyalkylene glycols such as polyethylene glycol, also 1,2-propanediol, 1,3-propanediol, butanediol(1,3), butanediol(1,4), hexanediol(1,6) and isomers, neopentyl glycol or neopentyl glycol hydroxypivalate, of which hexanediol(1,6) and isomers, neopentyl glycol and neopentyl glycol hydroxypivalate are preferred. Besides these it is also possible to use polyols such as trimethylol-propane, glycerol, erythritol, pentaerythritol, trimethylolbenzene or trishydroxyethyl isocyanurate.

Useful dicarboxylic acids include phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid. The corresponding anhydrides can also be used as a source of an acid.

When the average functionality of the polyol to be esterified is greater than 2, monocarboxylic acids, such as benzoic acid and hexanecarboxylic acid can also be used in addition.

Preferred acids are aliphatic or aromatic acids of the aforementioned kind. Adipic acid, isophthalic acid and optionally trimellitic acid are particularly preferred.

Hydroxycarboxylic acids useful as reaction participants in the preparation of a polyester polyol having terminal hydroxyl groups include for example hydroxycaproic acid, hydroxybutyric acid, hydroxydecanoic acid, hydroxystearic acid and the like. Suitable lactones are caprolactone, butyrolactone and homologs. Caprolactone is preferred.

I2) may likewise utilize hydroxyl-containing polycarbonates, preferably polycarbonate diols, having number average molecular weights Mn in the range from 400 to 8000 g/mol and preferably in the range from 600 to 3000 g/mol. These are obtainable by reaction of carbonic acid derivatives, such as diphenyl carbonate, dimethyl carbonate or phosgene, with polyols, preferably diols.

Examples of such diols are ethylene glycol, 1,2-propanediol, 1,3-propanediol, 1,3-butanediol, 1,4-butanediol, 1,6-hexanediol, 1,8-octanediol, neopentyl glycol, 1,4-bishydroxymethylcyclohexane, 2-methyl-1,3-propanediol, 2,2,4-trimethylpentane-diol-1,3, dipropylene glycol, polypropylene glycols, dibutylene glycol, polybutylene glycols, bisphenol A and lactone-modified diols of the aforementioned kind.

The polycarbonate diol preferably contains from 40 to 100 wt % of hexanediol, preferably 1,6-hexanediol and/or hexanediol derivatives, based on the underlying diols. Such hexanediol derivatives are based on hexanediol and have ester or ether groups as well as terminal OH groups. Such derivatives are obtainable by reaction of hexanediol with excess caprolactone or by etherification of hexanediol with itself to form di- or trihexylene glycol.

In lieu of or in addition to pure polycarbonate diols, polyether-polycarbonate diols can also be used in I2).

Hydroxyl-containing polycarbonates preferably have a linear construction.

I2) may likewise utilize polyether polyols. Useful polyether polyols include for example the well-known polyurethane chemistry polytetramethylene glycol polyethers obtainable by polymerization of tetrahydrofuran via cationic ring opening.

Useful polyether polyols likewise include the well-known addition products of styrene oxide, ethylene oxide, propylene oxide, butylene oxides and/or epichlorohydrin onto di- or polyfunctional starter molecules.

Useful starter molecules include all prior art compounds, for example water, butyl diglycol, glycerol, diethylene glycol, trimethylolpropane, propylene glycol, sorbitol, ethylenediamine, triethanolamine, 1,4-butanediol. Preferred starter molecules are water, ethylene glycol, propylene glycol, 1,4-butanediol, diethylene glycol and butyl diglycol.

It is particularly preferable for the polyurethanes to contain as component I2) a mixture of polycarbonate polyols and polytetramethylene glycol polyols wherein, in this mixture, the proportion of polycarbonate polyols is 20 to 80 wt % and the proportion of polytetramethylene glycol polyols is 80 to 20 wt %. Preference is given to a 30 to 75 wt % proportion of polytetramethylene glycol polyols and a 25 to 70 wt % proportion of polycarbonate polyols. Particular preference is given to a 35 to 70 wt % proportion of polytetramethylene glycol polyols and a 30 to 65 wt % proportion of polycarbonate polyols, each subject to the proviso that the sum total of the weight percentages for the polycarbonate and polytetramethylene glycol polyols is 100% and the proportion of component I2) which is accounted for by the sum total of the polycarbonate and polytetramethylene glycol polyether polyols is at least 50 wt %, preferably 60 wt % and more preferably at least 70 wt %.

I3) may utilize polyols of the recited molecular weight range that have up to 20 carbon atoms, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenylpropane), hydrogenated bisphenol A, (2,2-bis(4-hydroxycyclohexyl)propane), trimethylolpropane, glycerol, pentaerythritol and also any desired mixtures thereof with each or one another.

Also suitable are ester diols of the recited molecular weight range such as α-hydroxybutyl-ε-hydroxycaproic ester, ω-hydroxyhexyl-γ-hydroxybutyric ester, β-hydroxyethyl adipate or bis(β-hydroxyethyl) terephthalate.

I3) may further utilize monofunctional hydroxyl-containing compounds. Examples of such monofunctional compounds are ethanol, n-butanol, ethylene glycol monobutyl ether, diethylene glycol monomethyl ether, ethylene glycol monobutyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Preferred compounds of component I3) are 1,6-hexanediol, 1,4-butanediol, neopentyl glycol and trimethylolpropane.

An anionically or potentially anionically hydrophilicizing compound for component I4) is any compound which has at least one isocyanate-reactive group such as a hydroxyl group and also at least one functionality such as for example —COO-M⁺, —SO₃⁻M⁺, —PO(O-M⁺)² where M⁺ is for example a metal cation, H⁺, NH₄⁺, NHR₃⁺, where R in each occurrence may be C1-C12 alkyl, C5-C6 cycloalkyl and/or C2-C4 hydroxyalkyl, which functionality interacts with aqueous media by entering a pH-dependent dissociative equilibrium and thereby can have a negative or neutral charge. Suitable anionically or potentially anionically hydrophilicizing compounds are mono- and dihydroxycarboxylic acids, mono- and dihydroxysulfonic acids, and also mono- and dihydroxyphosphonic acids and salts thereof. Examples of such anionic or potentially anionic hydrophilicizing agents are dimethylolpropionic acid, dimethylolbutyric acid, hydroxypivalic acid, malic acid, citric acid, glycolic acid, lactic acid and the propoxylated adduct formed from 2-butenediol and NaHSO₃, as described in DE-A 2 446 440, page 5-9, formula I-III. Preferred anionic or potentially anionic hydrophilicizing agents for component I4) are those of the aforementioned kind that have carboxylate or carboxyl groups and/or sulfonate groups.

Particularly preferred anionic or potentially anionic hydrophilicizing agents for component I4) are those which contain carboxylate/carboxylic acid groups as ionic or potentially ionic groups, such as dimethylolpropionic acid, dimethylolbutyric acid and hydroxypivalic acid and salts thereof.

Suitable nonionically hydrophilicizing compounds for component I4) are for example polyoxyalkylene ethers that contain at least one hydroxyl or amino group, preferably at least one hydroxyl group. Examples are the monohydroxyl-functional polyalkylene oxide polyether alcohols containing on average from 5 to 70 and preferably from 7 to 55 ethylene oxide units per molecule and obtainable in a conventional manner by alkoxylation of suitable starter molecules (for example in Ullmanns Encyclopädie der technischen Chemie, 4^{th} edition, volume 19, Verlag Chemie, Weinheim pages 31-38).

These are either pure polyethylene oxide ethers or mixed polyalkylene oxide ethers, in which case they then contain at least 30 mol % and preferably at least 40 mol % of ethylene oxide units, based on all alkylene oxide units present.

Particularly preferred nonionic compounds are monofunctional mixed polyalkylene oxide polyethers having 40 to 100 mol % of ethylene oxide units and 0 to 60 mol % of propylene oxide units.

Suitable starter molecules for such nonionic hydrophilicizing agents are saturated monoalcohols such as methanol, ethanol, n-propanol, isopropanol, n-butanol, iso-butanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, for example diethylene glycol monobutyl ether, unsaturated alcohols such as allyl alcohol, 1,1-dimethylallyl alcohol or oleic alcohol, aromatic alcohols such as phenol, the isomeric cresols or methoxyphenols, araliphatic alcohols such as benzyl alcohol, anis alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine and also heterocyclic secondary amines such as morpholine, pyrrolidine, piperidine or 1H-pyrazole. Preferred starter molecules are saturated monoalcohols of the aforementioned kind. Particular preference is given to using diethylene glycol monobutyl ether or n-butanol as starter molecules.

Suitable alkylene oxides for the alkoxylation reaction are in particular ethylene oxide and propylene oxide, which can be used in the alkoxylation reaction in any desired order or else in admixture.

Component J1) may utilize di- or polyamines such as 1,2-ethylenediamine, 1,2-diaminopropane, 1,3-diaminopropane, 1,4-diaminobutane, 1,6-diaminohexane, isophoronediamine, isomeric mixture of 2,2,4- and 2,4,4-trimethylhexamethylene-diamine, 2-methylpentamethylenediamine, diethylenetriamine, triaminononane, 1,3-xylylenediamine, 1,4-xylylenediamine, α,α,α′,α′-tetramethyl-1,3-xylylenediamine, α,α,α′,α′-tetramethyl-1,4-xylylenediamine and 4,4-diaminodicyclohexylmethane and/or dimethylethylenediamine. It is likewise possible but less preferable to use hydrazine or and also hydrazides such as adipohydrazide.

Component J1) can further utilize compounds which as well as a primary amino group also have secondary amino groups or which as well as an amino group (primary or secondary) also have OH groups. Examples thereof are primary/secondary amines, such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanol-amine, 3-aminopropanol, neopentanolamine.

Component J1) can further also utilize monofunctional isocyanate-reactive amine compounds, for example methylamine, ethylamine, propylamine, butylamine, octyl-amine, laurylamine, stearylamine, isononyloxypropylamine, dimethylamine, diethyl-amine, dipropylamine, dibutylamine, N-methylaminopropylamine, diethyl(methyl)aminopropylamine, morpholine, piperidine, or suitable substituted derivatives thereof, amide-amines formed from diprimary amines and monocarboxylic acids, monoketimes of diprimary amines, primary/tertiary amines, such as N,N-dimethylaminopropylamine.

Preferred compounds for component J1) are 1,2-ethylenediamine, 1,4-diaminobutane and isophoronediamine.

An anionically or potentially anionically hydrophilicizing compound for component J2) is any compound which has at least one isocyanate-reactive group, preferably an animo group, and also at least one functionality such as for example —COO-M⁺, —SO₃⁻M⁺, —PO(O-M⁺)² where M⁺ is for example a metal cation, H⁺, NH₄⁺, NHR₃⁺, where R in each occurrence may be C1-C12 alkyl, C5-C6 cycloalkyl and/or C2-C4 hydroxyalkyl, which functionality interacts with aqueous media by entering a pH-dependent dissociative equilibrium and thereby can have a negative or neutral charge.

Suitable anionically or potentially anionically hydrophilicizing compounds are mono- and diaminocarboxylic acids, mono- and diaminosulfonic acids and also mono- and diaminophosphonic acids and salts thereof. Examples of such anionic or potentially anionic hydrophilicizing agents are N-(2-aminoethyl)-β-alanine, 2-(2-aminoethylamino)ethanesulfonic acid, ethylenediaminepropylsulfonic acid, ethylenediaminebutylsulfonic acid, 1,2- or 1,3-propylenediamine-β-ethylsulfonic acid, glycine, alanine, taurine, lysine, 3,5-diaminobenzoic acid and the addition product of IPDA and acrylic acid (EP-A 0 916 647, Example 1). It is further possible to use cyclohexylaminopropanesulfonic acid (CAPS) from WO-A 01/88006 as anionic or potentially anionic hydrophilicizing agent.

Preferred anionic or potentially anionic hydrophilicizing agents for component J2) are those of the aforementioned kind that have carboxylate or carboxylic acid groups and/or sulfonate groups, such as the salts of N-(2-aminoethyl)-β-alanine, of 2-(2-aminoethylamino)ethanesulfonic acid or of the addition product of IPDA and acrylic acid (EP-A 0 916 647, Example 1).

Mixtures of anionic or potentially anionic hydrophilicizing agents and nonionic hydrophilicizing agents can also be used for hydrophilicization.

It is further particularly preferable for the active substance to comprise a component that releases nitrogen monoxide under in vivo conditions, preferably L-arginine or an L-arginine-containing or an L-arginine-releasing component, more preferably L-arginine hydrochloride. Proline, ornithine and/or other biogenic intermediates such as for example biogenic polyamines (spermine, spermitine, putrescine or bioactive artificial polyamines) can also be used. Components of this type are known to augment wound healing, while their continuous substantially uniform rate of release is particularly conducive to wound healing.

Further active substances usable according to the present invention comprise at least one substance selected from the group of vitamins or provitamins, carotenoids, analgesics, antiseptics, hemostyptics, antihistamines, antimicrobial metals or salts thereof, plant-based wound healing promoter substances or substance mixtures, plant extracts, enzymes, growth factors, enzyme inhibitors and also combinations thereof.

Suitable analgesics are in particular non-steroidal analgesics especially salicylic acid, acetylsalicylic acid and its derivatives e.g. Aspirin®, aniline and its derivatives, acetaminophen e.g. Paracetamol®, anthranilic acid and its derivatives e.g. mefenamic acid, pyrazole or its derivatives e.g. methamizole, Novalgin®, phenazone, Antipyrin®, isopropylphenazone and most preferably arylacetic acids and derivatives thereof, heteroarylacetic acids and also derivatives thereof, arylpropionic acids and also derivatives thereof and heteroarylpropionic acids and also derivatives thereof e.g. Indometacin®, Diclophenac®, Ibuprofen®, Naxoprophen®, Indomethacin®, Ketoprofen®, Piroxicam®.

Suitable growth factors include in particular: aFGF (Acidic Fibroplast Growth Factor), EGF (Epidermal Growth Factor), PDGF (Platelet Derived Growth Factor), rhPDGF-BB (Becaplermin), PDECGF (Platelet Derived Endothelial Cell Growth Factor), bFGF (Basic Fibroplast Growth Factor), TGF α (Transforming Growth Factor alpha), TGF β (Transforming Growth Factor beta), KGF (Keratinocyte Growth Factor), IGF1/IGF2 (Insulin-Like Growth Factor) and TNF (Tumor Necrosis Factor).

Suitable vitamins or provitamins are especially the fat-soluble or water-soluble vitamins vitamin A, group of retinoids, provitamin A, group of carotenoids, especially β-carotene, vitamin E, group of tocopherols, especially α-tocopherol, β-tocopherol, γ-tocopherol, δ-tocopherol and α-tocotrienol, β-tocotrienol, γ-tocotrienol and δ-tocotrienol, vitamin K, phylloquinone especially phytomenadione or plant-based vitamin K, vitamin C, L-ascorbic acid, vitamin B1, thiamine, vitamin B2, riboflavin, vitamin G, vitamin B3, niacine, nicotinic acid and nicotinamide, vitamin B5, pantothenic acid, provitamin B5, panthenol or dexpanthenol, vitamin B6, vitamin B7, vitamin H, biotin, vitamin B9, folic acid and also combinations thereof.

A useful antiseptic is any antiseptic that has a germicidal, bactericidal, bacteriostatic, fungicidal, virucidal, virustatic and/or generally microbiocidal effect.

Antiseptic substances selected from the group resorcinol, iodine, iodine-povidone, chlorhexidine, benzalkonium chloride, benzoic acid, benzoyl peroxide or cetylpyridinium chloride are suitable in particular. In addition, antimicrobial metals in particular are also useful as antiseptics. Useful antimicrobial metals include in particular silver, copper or zinc and also their salts, oxides or complexes in combination or alone.

Plant-based active substances that promote wound healing in the context of the present invention are in particular extracts of chamomile, hamamelis extracts e.g. Hamamelis virgina, calendula extract, aloe extract e.g. Aloe vera, Aloe barbadensis, Aloe feroxoder or Aloe vulgaris, green tea extracts, seaweed extract e.g. red algae or green algae extract, avocado extract, myrrh extract e.g. Commophora molmol, bamboo extracts and also combinations thereof.

The silicone envelope which encapsulates the active substance particles may preferably consist of polydimethylsiloxane, polyvinylsiloxane, polyphenylsiloxane, polyalkylsiloxane, organomodified silicones such as for example polyether-polysiloxane copolymers, fluorosilicones or hydrosilicones, and more preferably of polydimethylsiloxane.

A refinement of the invention provides that the active substance depots are spherical and have a diameter of 10 to 2000, preferably of 100 to 1000 and more preferably of 300 to 850 μm. The diameter can be adjusted by sieving. Active substance depots of this type provide a particularly uniform release of the active substance ingredient throughout a period of 3 to 7 days.

The active substance content of the active substance depots can in particular be 2 to 60, preferably 2 to 50, more preferably 5 to 40 and even more preferably 5 to 30 wt %. This active substance content represents an optimum of particle formability, reservoir effect and release kinetics.

In an advantageous embodiment, the substrate contains from 0.1 to 20, preferably from 0.5 to 15, more preferably from 1 to 10 and even more preferably from 1 to 5 wt % of active substance depots, based on the total weight of the wound dressing. Wound dressings of this type display comparable mechanical and haptic properties to corresponding wound dressings without active substance depot and the external appearance of the wound dressing is not noticeably changed by the incorporated particles of silicone.

The invention further provides a wound dressing according to the invention for use as means for treating wounds.

The invention likewise provides for the use of a wound dressing according to the invention for producing a means for treating wounds.

### EXAMPLES

Unless indicated otherwise, all percentages are by weight.

Solids contents were determined in accordance with DIN-EN ISO 3251.

NCO contents, unless expressly mentioned otherwise, were determined volumetrically in accordance with DIN-EN ISO 11909.

Average particle sizes (the number average is reported) for polyurethane dispersion 1 were determined using laser correlation spectroscopy (instrument: Malvern Zetasizer 1000, Malver Inst. Limited).

Reported viscosities were determined by rotary viscometry to DIN 53019 at 23° C. using a rotary viscometer from Anton Paar Germany GmbH, Ostfildern, DE.

### SUBSTANCES AND ABBREVIATIONS USED

diaminosulfonate: NH₂—CH₂CH₂—NH—CH₂CH₂—SO₃Na (45% strength in water)
Desmophen® C2200: polycarbonate polyol, OH number 56 mg KOH/g, number average molecular weight 2000 g/mol (Bayer MaterialScience AG, Leverkusen, DE)
PolyTHF® 2000: polytetramethylene glycol polyol, OH number 56 mg KOH/g, number average molecular weight 2000 g/mol (BASF AG, Ludwigshafen, DE)
PolyTHF® 1000: polytetramethylene glycol polyol, OH number 112 mg KOH/g, number average molecular weight 1000 g/mol (BASF AG, Ludwigshafen, DE)
LB 25 polyether: monofunctional polyether based on ethylene oxide/propylene oxide, number average molecular weight 2250 g/mol, OH number 25 mg KOH/g (Bayer MaterialScience AG, Leverkusen, DE)
Arg: L-arginine (BASF AG, Ludwigshafen, DE)
Arg-HCl: L-arginine hydrochloride (Sigma, Steinheim, DE)
Sylgard® 184; comp. A: dimethylvinyl-terminated dimethylsiloxane (Dow Corning S.A., Seneffe, BE)
Sylgard® 184; comp. B: hydrosilane (Dow Corning S.A., Seneffe, BE)
Syl-Off® 4000: organoplatinum catalyst (Dow Corning S.A., Seneffe, BE)
Pluronic® F127: EO-PO block copolymer (BASF, Ludwigshafen, DE)
Pluronic® PE6800: EO-PO block copolymer (BASF, Ludwigshafen, DE)
Desmodur® N 3400: aliphatic polyisocyanate (HDI uretdione), NCO content 21.8% (Bayer MaterialScience AG, Leverkusen, DE)
DBU: 1,8-diazabicyclo(5.4.0)undec-7-ene (Sigma, Steinheim, DE)

### Example 1

### Preparation of Polyurethane Dispersion 1

1077.2 g of PolyTHF® 2000, 409.7 g of PolyTHF® 1000, 830.9 g of Desmophen® C2200 and 48.3 g of LB 25 polyether were heated to 70° C. in a standard stirred apparatus. Then, a mixture of 258.7 g of hexamethylene diisocyanate and 341.9 g of isophorone diisocyanate was added at 70° C. in the course of 5 min before stirring at 120° C. until the theoretical NCO value was reached or the actual NCO value had dropped to slightly below the theoretical NCO value. The ready-produced prepolymer was dissolved with 4840 g of acetone and, in the process, cooled down to 50° C. and subsequently admixed with a solution of 27.4 g of ethylenediamine, 127.1 g of isophoronediamine, 67.3 g of diaminosulfonate and 1200 g of water by the solution being metered in over 10 min. The mixture was subsequently stirred for 10 min. Then, a dispersion was formed by addition of 654 g of water. The solvent was subsequently removed by distillation under reduced pressure.

The polyurethane dispersion obtained had the following properties:

Solids content: 62.2%

Particle size (LCS): 528 nm

pH (23° C.): 7.5

### Example 2

### Preparation of Silicone Particles 1

The particular amount of Arg or Arg-HCl crystals (see table 1) was homogeneously dispersed in 8.55 g of component A of Sylgard® 184 and 0.34 g of Syl-Off® 4000. Any air bubbles were removed by ultrasonication and evacuation. Then, 0.85 g of component B of Sylgard® and 3.125 mL of chloroform were added. After carefully mixing the suspension, it was added dropwise at 25° C. during 6-10 minutes via syringe and 15 G canula into a mixture formed from 300 ml of a 13.3% strength Pluronic® F127 solution in water and 700 mL of methanol and saturated with Arg-HCl (stirrer speed of methanolic solution: 350 rpm). On completion of the addition the mixture was stirred at 25° C. at 300 rpm for 2 hours and for a further 30 min at 50° C. Finally, the silicone particles of the cooled-down suspension were separated with metal sieves into three fractions (ø≦300 μm; 300 μm≦0≦850 μm; ø≧850 μm) and washed with water and methanol. The particles were conditioned overnight at 50° C. under reduced pressure.

TABLE 1

| | Arginine | Amount | Arg fraction* |
|---|---|---|---|
| # | source | added [g] | [wt %] |
| 1 | Arg-HCl | 0.63 | 5.0 |
| 2 | Arg-HCl | 1.33 | 9.9 |
| 3 | Arg-HCl | 2.16 | 15.0 |
| 4 | Arg-HCl | 3.13 | 20.1 |
| 5 | Arg | 1.08 | 9.9 |

*arginine fraction based on resulting overall mass of particle

### Example 3

### Production of Foams from Polyurethane Dispersion 1 and Silicone Particles 1

100 g of polyurethane dispersion 1, prepared according to Example 1, were mixed with 2.31 g of silicone particles 1 (300 μm≦ø≦850 μm, obtained by sieving) containing 20% of Arg using a commercially available hand stirring implement (stirrer made of bent wire). After subsequent addition of 11.3 g of a 30% strength solution of Pluronic® PE 6800 in water, the mixture was frothed up to a 500 ml foam volume. Thereafter, the foams were drawn down on non-stick paper using a film applicator (blade coater) set to a gap height of 5 mm and dried at 100° C. in a circulating air drying cabinet for 45 min.

A clean white foam was obtained with homogeneously dispersed particles of silicone and good mechanical properties.

Release tests were carried out with 1.5 g of this foam, which contained 3.4% of silicone particles 1 or 0.7% of Arg, at 20° C. using a diffusion cell (flow rate about 1.1 ml/h; guided by the customary lymph flux) using phosphate-buffered saline solution (pH 7.4).

FIG. 1 shows the non-cumulative concentrations of released arginine: The therapeutically necessary Arg concentration of 100 μmol/L is reached within a few hours and maintained for about 3 days, which corresponds to the customary residence time of a wound dressing on a corresponding wound. The active substance reservoir is still not exhausted after 5-7 days.

### Example 4

### Producing a Foam-Silicone Particle Composite Material from Polyurethane Dispersion 1 and Silicone Particles 1

100 g of polyurethane dispersion 1, prepared as described in Example 1, were mixed with 9.9 g of a 30% strength solution of Pluronic® PE 6800 in water and a commercially available hand stirring implement (stirrer made of bent wire) was used to froth up the mixture to 400 mL foam volume. The foam was then drawn down on non-stick paper by means of a film applicator (blade coater) at a gap height of 2 mm and, while still moist, was sprinkled with 6.37 g of silicone particles 1, prepared as described in Example 2. A further layer of foam was then applied atop the first, still moist layer of foam using a film applicator such that the silicone particles became encased from both sides by polyurethane foam. The composite material was dried at 120° C. in a circulating air drying cabinet for 20 minutes.

Clean white foam-silicone particle composite materials were obtained with good mechanical properties (peel strength≧0.8 N/mm) and a fine porous structure. The silicone particles were at a position centrally in the interface between the two layers of foam.

The peel strength was determined on a Zwick universal tester. For this, the two layers of foam were peeled apart at an angle of 180° at a traverse speed of 100 mm/min.

### Example 5

### Producing a Foam-Silicone Particle Composite Material from Polyurethane Dispersion 1 and Silicone Particles 1

First, a commercially available hand stirring implement (stirrer made of bent wire) was used to mix 100 g of polyurethane dispersion 1, prepared as described in Example 1, with 9.9 g of a 30% strength solution of Pluronic® PE 6800 in water and froth up the mixture to 400 mL foam volume. The foam was then drawn down on non-stick paper using a film applicator (blade coater) set to a gap height of 2 mm and dried at 140° C. in a circulating air drying cabinet for 15 min. The silicone particles 1 prepared as described in Example 2 were then sprinkled onto this dried foam in the form of islands, covered with a further layer of foam and the two layers of foam were compressed together at 160° C. for 60 s to a thickness of 1 mm, i.e., 25% of the original thickness, such that the silicone particles became fully encased in foam in the resulting foam pocket.

Clean white foam-silicone particle composite materials were obtained with good mechanical properties (peel strength≧0.8 N/mm) and a fine porous structure. The silicone particles were enclosed in pocket fashion between the two layers of foam.

### Example 6

### pH-Dependent Arginine Release from Silicone Particles/PU Foams

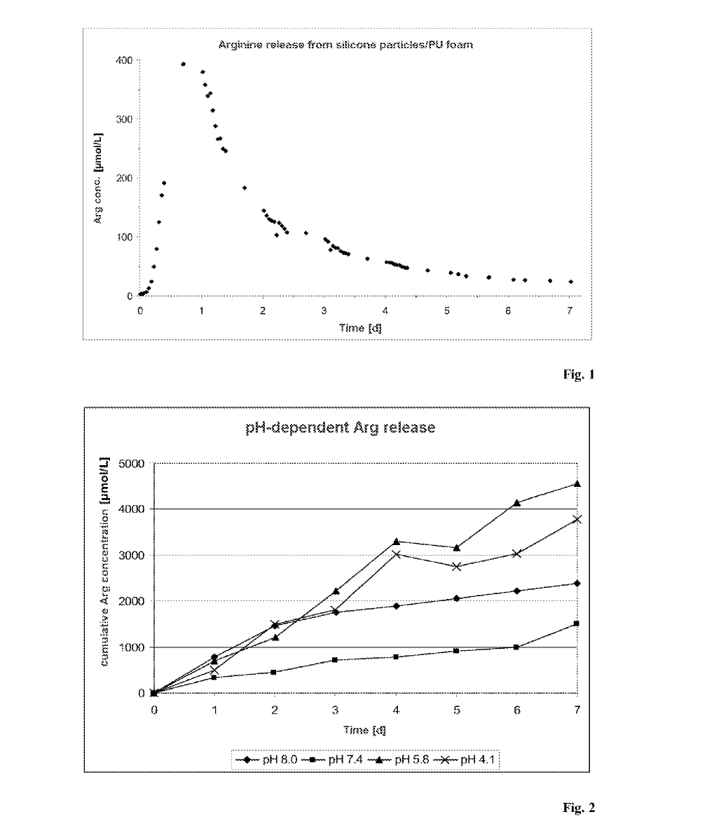

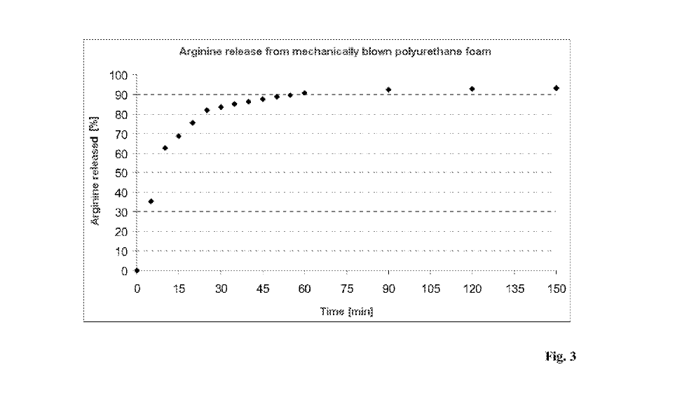

In the course of the healing process of a chronic wound, for example, the pH of the wound milieu will also often change as wound healing progresses. For this reason, release tests as per Example 3 were carried out with PBS buffer solutions having different pH values. The resulting release profiles, shown in FIG. 2, illustrate that the pH—particularly because of the pH-dependent Arg solubility—has an influence on the release behavior of the active substance. The depicted Arg concentrations of a series of measurements are in each case cumulative values.

### Example 7

### Producing a Polyurethane Reactive Foam Comprising Silicone Particles

A mixture of 1440 g of HDI and 4 g of benzoyl chloride was admixed at 80° C., by dropwise addition over 2 h, with 2880 g of a polyalkylene oxide having a molar mass of 4680 g/mol started on glycerol, an ethylene oxide weight fraction of 72% and a propylene oxide weight fraction of 28%, dried beforehand at 100° C. for 6 h at a pressure of 0.1 mbar, and subsequently stirred for 1 h. Excess HDI was removed by thin film distillation at 130° C. and 0.1 mbar to obtain a prepolymer having an NCO content of 2.11% and a viscosity of 3780 mPas.

20.0 g of this prepolymer and 2.2 g of Desmodur® N 3400 were homogenized for 15 seconds at a stirrer speed of 1200 rpm, admixed with 2.2 g of silicone particles 1 and again homogenized at a low speed. Following addition of 0.08 g of DBU and 11.1 g of a 1% strength sodium oleate solution in water, the mixture was stirred for a further 10 seconds and then introduced into a beaker having a capacity of 500 ml. Following a cream time of about 20 s, the mixture foams up to form, within a few minutes, a dimensionally stable, elastic foam of regularly fine cellular structure with homogeneously dispersed particles of silicone in the interior.

The release tests performed corresponded to the results as per Example 3 not only as regards procedure but also as regards the resulting release profile.

### Comparative Example 1

### Arginine Release from a Mechanically Blown Polyurethane Foam

A commercially available hand stirring implement (stirrer made of bent wire) was used to mix 120 g of polyurethane dispersion 1, prepared as described in Example 1, with 12.4 g of a 30% strength solution of Pluronic® PE 6800 in water and 1.5 g of a 50% strength solution of Arg-HCl in water and froth up the mixture to 500 mL foam volume. The foam was then applied with a film applicator (blade coater) at 6 mm gap height to non-stick paper and dried at 140° C. in a circulating air drying cabinet for 15 min.

The result was a clean white foam having an Arg-HCl content of 0.95% based on overall mass, which corresponds to an arginine content of 0.78%.

The release tests were carried out with 1.3 g of this foam which, at 22° C., using a Franz diffusion cell was merely in surface contact with 110 g of phosphate-buffered saline (pH 7.4). FIG. 3 shows the cumulative amount of released arginine: above 60% of the arginine is released during the first 10 minutes; after 30 minutes, the active substance reservoir is almost exhausted.

### Comparative Example 2

### Producing an Arginine-Containing Reactive Polyurethane Foam

20.0 g of the prepolymer prepared as described in Example 6 and 2.2 g of Desmodur® N 3400 were homogenized for 15 seconds at a stirrer speed of 1200 rpm, admixed with 0.23 g of Arg-HCl and again homogenized at a low speed. Following addition of 0.08 g of DBU and 11.1 g of a 1% strength sodium oleate solution in water, the mixture was stirred for a further 10 seconds and then introduced into a beaker having a capacity of 500 ml. Following a cream time of about 30 s, the mixture foams up slowly, but is still not fully cured 30 minutes later; the introduced Arg-HCl accordingly has an adverse effect on the foaming reaction. The incompletely and inhomogeneously foamed mass was not subjected to any release tests, since no suitable foam resulted.

1-13. (canceled)

14. A wound dressing comprising a liquid-absorbing substrate having active substance depots contained therein, wherein the active substance depots comprise particles of at least one active substance which are encapsulated in a silicone envelope, and the substrate is a foam.

15. The wound dressing as claimed in claim 14, characterized in that the foam has a density of ≦0.5, preferably of ≦0.4, more preferably of ≧0.01 to ≧0.3 and even more preferably of ≧0.05 to ≦0.3 g/cm3.

16. The wound dressing as claimed in claim 14, wherein the foam is a polymer-based foam, preferably a reactive foam or a mechanically blown foam.

17. The wound dressing as claimed in claim 14, wherein the foam is based on polyurethanes.

18. The wound dressing as claimed in claim 17, wherein the polyurethanes are obtainable by reaction of
A) isocyanate-functional prepolymers having a weight fraction of low molecular weight aliphatic diisocyanates having a molar mass of 140 to 278 g/mol of below 1.0 wt %, based on the prepolymer, obtainable by reaction of
A1) low molecular weight aliphatic diisocyanates having a molar mass of 140 to 278 g/mol with
A2) di- to hexafunctional polyalkylene oxides having an OH number of 22.5 to 112 mg KOH/g and an ethylene oxide content of 50 to 100 mol %, based on the total amount of oxyalkylene groups present,
B) optionally heterocyclic 4-ring or 6-ring oligomers of low molecular weight aliphatic diisocyanates having a molar mass of 140 to 278 g/mol,
C) water,
D) optionally catalysts,
E) C₈-C₂₂ monocarboxylic acids or their ammonium or alkali metal salts or C₁₂-C₄₄ dicarboxylic acids or their ammonium or alkali metal salts,
F) optionally surfactants,
G) optionally mono- or polyhydric alcohols, and
H) optionally hydrophilic polyisocyanates obtainable by reaction of
H1) low molecular weight aliphatic diisocyanates having a molar mass of 140 to 278 g/mol and/or polyisocyanates obtainable therefrom with an isocyanate functionality of 2 to 6, with
H2) monofunctional polyalkylene oxides having an OH number of 10 to 250 and an ethylene oxide content of 50 to 100 mol %, based on the total amount of oxyalkylene groups present.

19. The wound dressing as claimed in claim 17, wherein the polyurethanes are obtainable by
I) isocyanate-functional prepolymers being produced at least from
I1) organic polyisocyanates,
I2) polymeric polyols having number average molecular weights of 400 to 8000 g/mol and OH functionalities of 1.5 to 6, and
I3) optionally hydroxyl-functional compounds having molecular weights of 62 to 399 g/mol, and
I4) optionally isocyanate-reactive, anionic or potentially anionic and/or optionally nonionic hydrophilicizing agents,
J) their free NCO groups then being wholly or partly reacted
J1) optionally with amino-functional compounds having molecular weights of 32 to 400 g/mol and/or
J2) with isocyanate-reactive, preferably amino-functional, anionic or potentially anionic hydrophilicizing agents
by chain extension, and the prepolymers being dispersed in water before, during or after step J), any potentially ionic groups present being converted into the ionic form by partial or complete reaction with a neutralizing agent.

20. The wound dressing as claimed in claim 14, wherein the active substance comprises a component that releases nitrogen monoxide under in vivo conditions, preferably L-arginine or an L-arginine-containing or an L-arginine-releasing component, more preferably L-arginine hydrochloride.

21. The wound dressing as claimed in claim 14, wherein the silicone envelope consists of polydimethylsiloxane.

22. The wound dressing as claimed in claim 14, wherein the active substance depots are spherical and have a diameter of 10 to 2000, preferably of 100 to 1000 and more preferably of 300 to 850 μm.

23. The wound dressing as claimed in claim 14, wherein the active substance content of the active substance depots is 2 to 60, preferably 2 to 50, more preferably 5 to 40 and even more preferably 5 to 30 wt %.

24. The wound dressing as claimed in claim 14, wherein the substrate contains from 0.1 to 20, preferably from 0.5 to 15, more preferably from 1 to 10 and even more preferably from 1 to 5 wt % of active substance depots, based on the total weight of the wound dressing.

25. The wound dressing as claimed in claim 14 for use as means for treating wounds.

26. The use of a wound dressing according to claim 14 for producing a means for treating wounds.
